(19) United States
(12) Patent Application Publication
(10) Pub. No.: US 2006/0178588 A1
(43) Pub. Date: Aug. 10, 2006

(54) System and method for isolating effects of basal autonomic nervous system activity on heart rate variability

(76) Inventor: Lee Brody, Somerville, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/315,080

(22) Filed: Dec. 22, 2005

Related U.S. Application Data
(60) Provisional application No. 60/640,862, filed on Jan. 3, 2005.

(51) Int. Cl. A61B 5/04

(52) U.S. Cl. 600/513

(57) ABSTRACT

Disclosed herein is an apparatus, method, and system that can be used to (1) simultaneously monitor HRV and other physiological parameters, (2) determine whether HRV can be reliably collected, (3) detect ANS arousal, (4) determine the effects of the ANS arousal on HRV, (5) determine the minimum extent to which a timed HRV study should be extended after an ANS arousal, and (6) analyze the effects of ANS arousal on HRV.

### CROSS-REFERENCE TO RELATED APPLICATIONS

This application claims the benefit of U.S. Application No. 60/640,862 filed Dec. 29, 2005, the disclosure of which is incorporated in its entirety.

### BACKGROUND OF THE INVENTION

1. Field of the Invention

This invention relates generally to the measurement of Heart Rate Variability (HRV), and more particularly, to a method for isolating the effects of basal autonomic nervous system (ANS) activity on HRV.

2. Description of the Related Art

HRV refers to the beat-to-beat alterations in heart rate. One method for measuring heart rate, and consequently, HRV, is to measure the interval between R waves in an electrocardiogram (ECG). Under resting conditions, the ECG exhibits periodic variation in R-R intervals. These alterations reflect the basal ANS modulation of heart rate. This information is of great interest to many clinicians because it provides specific insight into how a patient's ANS controls involuntary body functions at rest.

ANS arousal can result in a change in how the ANS controls involuntary body functions, however, including HRV. ANS arousal can be caused by both physical and emotional activity. The challenge for those clinicians and scientists looking to isolate the effects of basal ANS activity on HRV is unanticipated and undetected ANS arousal.

Generally speaking, there are two techniques that are utilized to isolate the effects of basal ANS activity on HRV; (1) collect HRV data over an extended period of time and (2) attempt to control the physical and emotional state of a patient during data collection.

With regard to extended periods of data collection, many healthcare professionals will opt to collect HRV data over a 24-hour period to “filter out” the fluctuations in HRV due to ANS arousal. This data collection technique is often time consuming and expensive.

In order to control physical and emotional states, healthcare professionals sometimes opt to collect HRV data over a short period of time (typically five minutes), and attempt to eliminate ANS arousal through verbal instruction. The goal is to elicit a “physiologic quieting” state in which there is no ANS arousal due to physical or emotional activity. This data collection technique is susceptible to undetected ANS arousal particularly from emotional activity.

### SUMMARY OF THE INVENTION

An aspect of at least one of the embodiments disclosed herein includes the realization that ANS arousal is detectable independently of HRV, and as such, the effects of ANS arousal on HRV is determinable through the monitoring of HRV and at least one other physiological parameter.

Disclosed herein is an apparatus, method, and system for (1) simultaneously monitoring HRV and other physiological parameters, (2) determining whether HRV can be reliably collected, (3) detecting ANS arousal, (4) determining the effects of the ANS arousal on HRV, (5) determining the minimum extent to which a timed HRV study should be extended after an ANS arousal, and (6) analyzing the effects of ANS arousal on HRV.

Accordingly, some embodiments provide an apparatus for measuring heart rate variability comprising: a heartbeat detector; and a detector configured to detect a physiological property that affects a heart rate variability measurement. Some embodiments further comprise a data processor configured to accept data from the heartbeat detector and the detector for a physiological property that affects a heart rate variability measurement, and to calculate a heart rate variability from these data.

Other embodiments provide an apparatus for determining heart rate variability comprising: means for measuring heart rate variability; means for measuring at least one physiological signal, wherein the physiological signal is a reliability indicator for the heart rate variability measurement, an indicator of autonomic nervous system activity, or a combination thereof; and means for integrating the heart rate variability measurement with the measurement of the at least one physiological signal.

Other embodiments provide a method for determining heart rate variability comprising: collecting a set of heartbeat data; detecting a physiological property related to the reliability of a heart rate variability measurement; and calculating a heart rate variability from the heartbeat data if the physiological property related to the reliability of a heart rate variability measurement indicates that the heart rate variability measurement is reliable.

Other embodiments provide a method for detecting autonomic nervous system arousal comprising: acquiring a first measurement of autonomic nervous system activity; acquiring second measurement of autonomic nervous system activity; and comparing first and second measurements of autonomic nervous system activity.

Other embodiments provide a method for determining the effect of autonomic nervous system arousal on heart rate variability comprising: collecting a first set of heartbeat data; acquiring a first measurement of autonomic nervous system activity; optionally inducing autonomic nervous system arousal; collecting a second set of heartbeat data; acquiring a second measurement of autonomic nervous system activity; comparing the first and second measurements of autonomic nervous system activity; calculating a heart rate variabilities from the first and second sets of heartbeat data; and determining the effect of autonomic nervous system arousal on heart rate variability. Some embodiments further comprise: collecting at least one additional set of heartbeat data; acquiring at least one additional measurement of autonomic nervous system activity; calculating a heart rate variability from the at least one additional set of heartbeat data; and comparing the at least one additional measurement of autonomic nervous system activity with a previous measurement of autonomic nervous system activity. Some embodiments further comprise determining the recovery of heart rate variability from autonomic nervous system arousal. Some embodiments further comprise determining a baseline heart rate variability. Some embodiments further comprise determining a correction for a heart rate variability measurement taken during ANS arousal.

Other embodiments provide a method for monitoring heart rate variability comprising: measuring a heart rate variability; measuring a physiological signal substantially simultaneously with the step of measuring the heart rate variability, wherein the physiological signal comprises at least one of reliability indicators for the reliability of the heart rate variability measurement and indicators of autonomic nervous system arousal; and integrating the heart rate variability measurement with the measurement of the physiological signal.

Other embodiments provide a method for determining the effect of autonomic nervous system arousal on heart rate variability comprising: substantially simultaneously acquiring a first heart rate variability measurement and a first measurement of an indicator of autonomic nervous system arousal; substantially simultaneously acquiring a second heart rate variability measurement and a second measurement of the indicator of autonomic nervous system arousal; comparing the first and second heart rate variability measurements; comparing the first and second heart measurements of the indicator of autonomic nervous system arousal; and determining the effect of the indicator of autonomic nervous system arousal on heart rate variability measurement.

Other embodiments provide a method for measuring a heart rate variability in a subject comprising: substantially simultaneously acquiring a first heart rate variability measurement and a first measurement of an indicator of autonomic nervous system arousal, wherein the measurements are taken in a non-aroused state of the autonomic nervous system; inducing autonomic nervous system arousal; substantially simultaneously acquiring a second heart rate variability measurement and a second measurement of the indicator of autonomic nervous system arousal; comparing the first and second heart rate variability measurements; comparing the first and second heart measurements of the indicator of autonomic nervous system arousal; and using the comparisons to derive a heart rate variability measurement.

### BRIEF DESCRIPTION OF THE DRAWINGS

FIG. 1 is an ECG illustrating variations in heart rate.

FIG. 2 is a schematic block diagram illustrating the relationship between HRV and the sympathetic and parasympathetic branches of the autonomic nervous system.

FIG. 3A is a block diagram illustrating an embodiment of an apparatus for monitoring HRV and another physiological parameter.

FIG. 3B illustrates a commercial embodiment of an apparatus constructed in accordance with an embodiment.

FIG. 3C illustrates a modification of the embodiment of FIG. 3B.

FIG. 4 is a flowchart of an embodiment of a method that can be used for determining HRV.

FIG. 5 is a flowchart of an embodiment of a method that can be used for detecting ANS arousal.

FIG. 6 is a flowchart of an embodiment of a method that can be used for determining the effect of ANS arousal on HRV.

FIGS. 7A-7C illustrate exemplary time series of instantaneous heart rate and skin conductance using the apparatuses illustrated in FIGS. 3B and 3C.

FIG. 8 illustrate the VLF, LF, and HF bands from exemplary heartbeat data.

### DETAILED DESCRIPTION OF THE PREFERRED EMBODIMENT

Systems and methods for isolating effects of basal autonomic nervous system activity on heart rate variability are disclosed below in the context of measuring an accurate HRV value with reference to drawings. The systems and methods are described in the context of measuring an accurate HRV value because they have particular utility in this context. However, the systems and methods disclosed herein can be used in other contexts, such as, for example, monitoring the balance between the two branches of the autonomic nervous system.

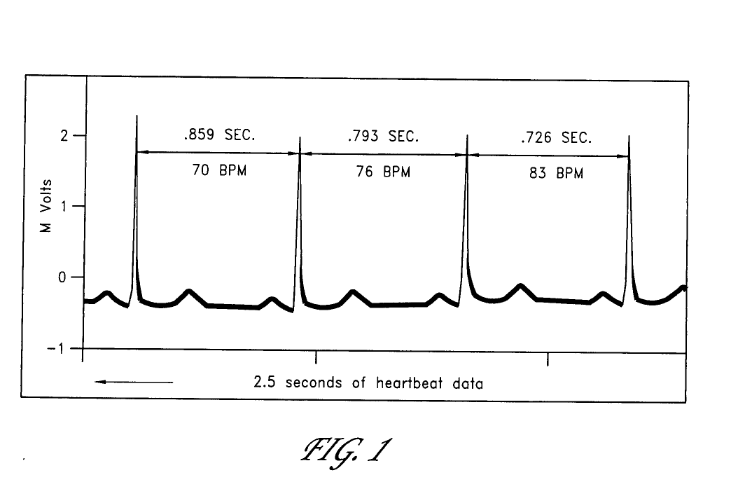

FIG. 1 illustrates an ECG showing three complete typical heartbeat cycles and the variations between the R-waves (interbeat intervals, hereinafter “IBI”) indicated in seconds and in beats/min. HRV quantifies the variability of the IBIs, and as such, is expressible either in the time domain or in the frequency domain. In the time domain, HRV is typically expressed as the standard deviation of the IBI in milliseconds. Analysis of HRV in the frequency domain permits differentiation between the contributions of the two branches of the autonomic nervous system, as discussed below. For example, in a five minute data recording, the total energy for of the variability can range from about 200 ms² to about 10000 ms² over a relevant frequency range of from about 0.003 to about 0.4 Hz.

Decreased HRV has been correlated with decreased adaptability, increased mortality risk, and increased risk for arrhythmic events. For example, HRV can be depressed in individuals with coronary artery disease, obesity, and/or exposure to air pollution. HRV also tends to decrease with age. Meditation and exercise, for example, tai chi and yoga, appear to counteract this decrease.

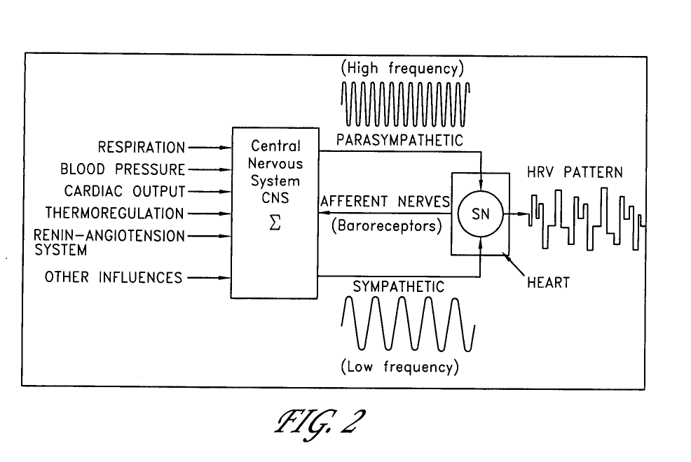

As reflected in the schematic block diagram of FIG. 2, HRV is believed to reflect the interaction between the sympathetic and parasympathetic branches of the autonomic nervous system in humans. Accordingly, HRV can be useful in assessing the relative balance between the sympathetic and parasympathetic nervous systems

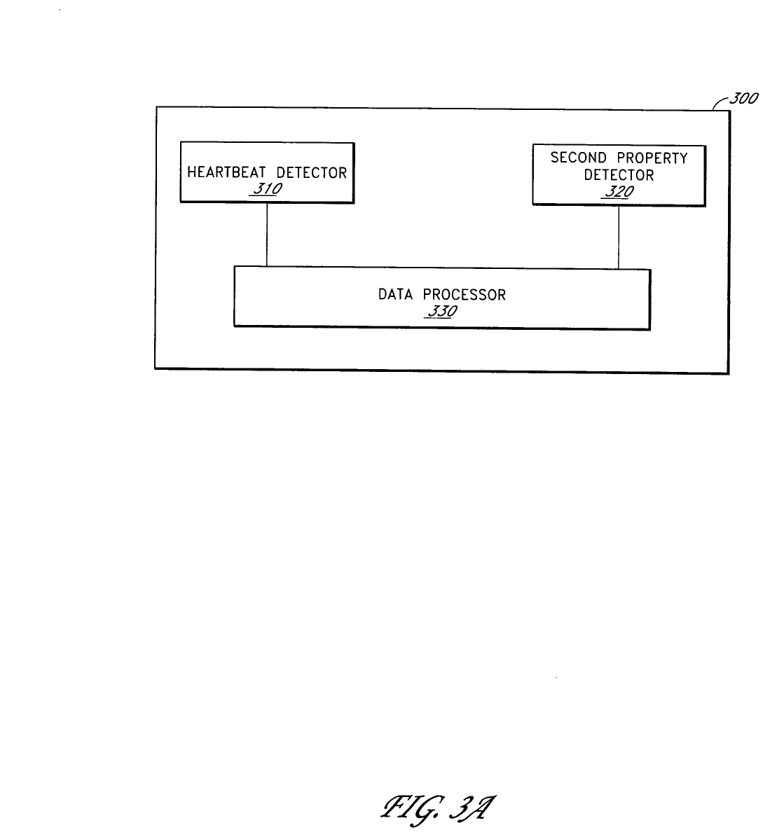

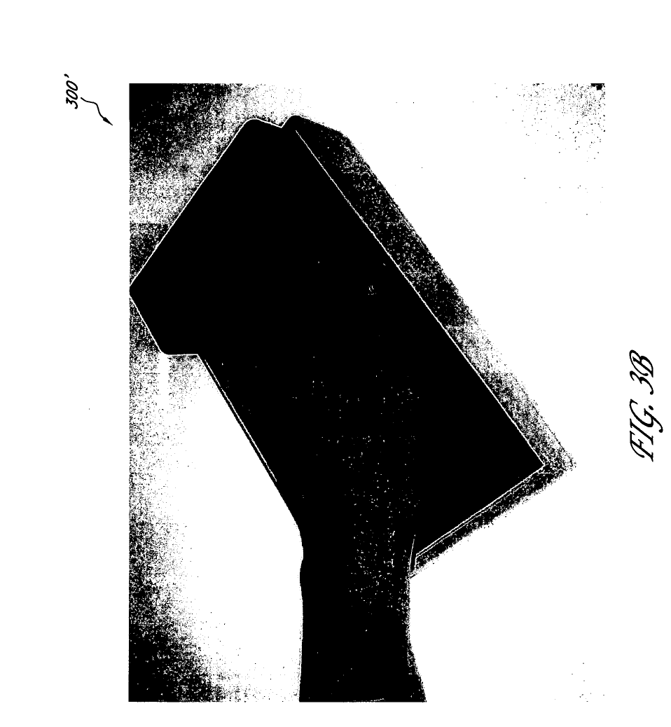

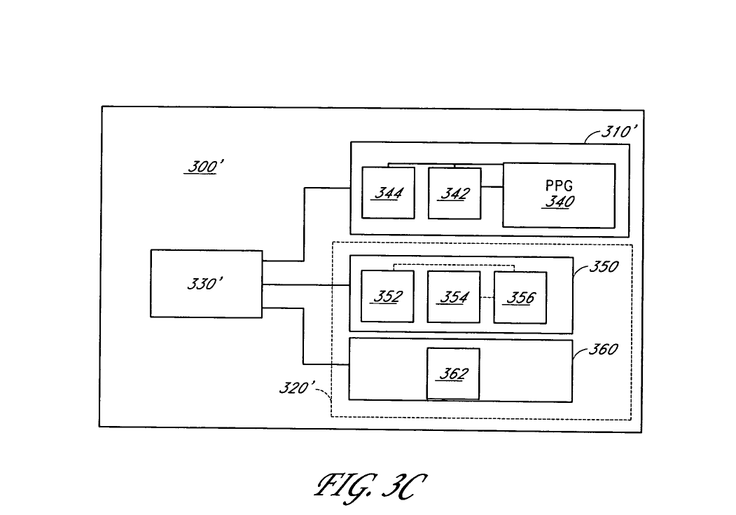

FIG. 3A is a schematic block diagram illustrating an HRV monitor 300 that can be configured to monitor HRV and/or other physiological parameters. The monitor 300 can comprise a heartbeat module 310, an HRV detection module 320, which can be configured to detect a physiological property that affects HRV, and a data processor 330 configured to accept data streams from the heartbeat detector and the detector for a physiological property that affects HRV, and to perform data analysis of the data streams. However, other configurations, features, and components configured to provide the same and/or additional functions can also be used.

The heartbeat module 310 can be any device known in the art for detecting a heartbeat and/or a pulse, for example, an electrocardiograph (ECG) machine, a photoelectric plethysmograph (PPG), an audio sensor, a pressure transducer, an ultrasonic imager, or the like. In some preferred embodiments, the heartbeat module 310 can be a photoelectric plethysmograph. Some embodiments of the monitor 300 use a plurality of heartbeat detectors, either of the same type and/or of different types.

The HRV detection module 320 can be configured to detect a physiological property that affects HRV, and can be referred to as a “second property detector.” The second property detector 320 can be configured to detect at least one physiological property that affects an HRV measurement.

Physiological properties that affect HRV measurements generally fall into two categories; (1) physiological properties that indicate the reliability of an HRV measurement, and (2) physiological properties reflecting ANS activity. Examples of physiological properties that indicate the reliability of an HRV measurement are known in the art and can include skin temperature and respiration rate. However, other physiological properties can also be used.

Examples of physiological properties reflecting ANS activity are known in the art and can include, for example, skin conductance level (SCL) and skin temperature. However, other physiological properties can also be used. Additional parameters are discussed in greater detail below. Those skilled in the art will understand that some physiological properties fall into both categories. Particular devices for measuring these properties depend on the particular property detected and are known in the art. In some embodiments, the at least one other physiological signal is at least one of a reliability indicator for HRV, an indicator of ANS arousal, or any combination thereof.

The data processor 330 can be any type known in the art. For example, the data processor 330 can comprise a microprocessor, computer, embedded processor, a hard-wired device, or the like configured to perform the functions noted below, and/or other functions. In some embodiments, the data processor is configured to accept substantially contemporaneous data streams from the heartbeat module 310 and the second property detector 320. In some embodiments the data processor 330 can be configured to perform additional functions, for example, providing a user interface, providing a machine interface, performing diagnostics, and the like. Examples of user interface elements include instructions to the user, menus for selecting different types of measurements, display of the results of a measurement, and the like.

FIG. 3B illustrates a modification of the HRV monitor 300, identified generally by the reference numeral 300′. A commercial embodiment of the monitor 300′, also referred to as a multimodality physiologic monitor, is available under the trade name Insight Pulse Wave Profiler (CLA, Mahwah, N.J.). The monitor 300′ can be configured to simultaneously measure HRV, skin conductance level (SCL), skin temperature, and/or other parameters. In such embodiments, the second property detector 320′ is configured to detect SCL and skin temperature, described in greater detail below.

With regard to the measuring of HRV, the illustrated monitor 300′ can include a heartbeat monitor 310′ which can comprise a photoelectric plethysmograph (PPG) 340 and an emitter/detector pair 342, 344 configured to be placed in contact with a middle finger, or other finger, of a human subject. In embodiments using a PPG, the PPG 340 can be configured to detect the pulsatile volume changes of blood pooling in the capillaries. Signal processing techniques, known in the art, allow for the determination of successive R-R intervals and thus HRV. For example, the processor 330′ can be configured to perform such known signal processing techniques to determine successive R-R intervals, the variation of such intervals, and thus generate a value indicative of HRV.

With regard to the detection of skin conductance level (SCL), the monitor second property detector 320 can include a skin conductance level module 350, configured to detect and generate a value or signal indicative of the skin conductance level of a subject. For example, in some embodiments, the SCL module 350 can comprise electrodes 350, 352, which in some embodiments can be gold plated, configured to be placed into contact with one or more fingers to generate a value indicative of skin conductance. In some embodiments, the electrodes 350, 352 can be disposed or orientated for contact with both the index and ring fingers, or other fingers, of a human subject. Standard signal processing techniques allow for the calculation of real-time skin SCL measured in microSiemens. For example, in embodiments where the SCL module 350 comprises only the electrodes 352, 354, and an optional analog-digital converter (not shown), the processor 330′ can be configured to sample the output of the analog-to-digital converter, to generate an SCL value. In some embodiments, the SCL module 350 can include an optional dedicated processor 356 for sampling the electrodes 352, 354 and generating a value indicative of SCL.

With regard to the detection of skin temperature, the second property detector 320 can include a temperature sensing module 360. In some embodiments, the temperature sensor can include a thermistor 362 is placed in direct contact with the middle finger, or any other finger or part of a human subject. However, any type of temperature sensor can also be used. Standard signal processing techniques can be used to calculate values indicative of the skin temperature of the subject. Optionally, this determination can be performed in real-time.

The sensors 344, 342, 352, 354, 362 can be arranged and connected to any portion of the anatomy of the subject patient. In the embodiment illustrated in FIG. 3B, the monitor 300′ includes a housing configured to support a left or right hand of a human patient. The sensors 344, 342, 352, 354, 362 can be mounted to the housing so as to contact one of the index, middle, and ring fingers of the human subject. However, the sensors 344, 342, 352, 354, 362 can be arranged in other configurations so as to make contact with other combinations of the subject's fingers. FIG. 3D illustrates yet another modification of the monitor 300, identified generally by the reference numeral 300″. The monitor 300″ can have the same or similar construction as the monitor 300′.

Those skilled in the art will understand other devices known in the art are also useful for measuring these physiological signals. In other embodiments, additional physiological signals, for example, respiration rate, are measured. Other embodiments comprise detectors for a plurality of physiological properties that affect HRV.

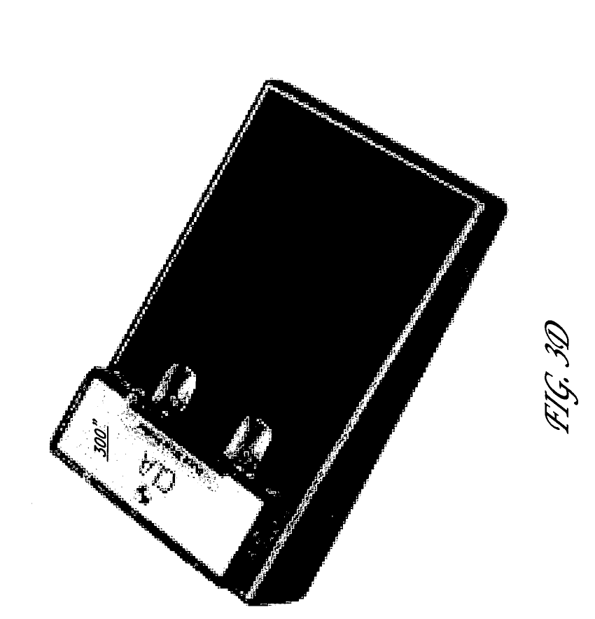

FIG. 4 is a flow chart illustrating an embodiment of a method 400 for determining HRV that can be used with any of the monitors 300, 300′, 300″, illustrated in FIGS. 3A, 3B, and 3C, but those skilled in the art will understand that the method is also useful with other devices.

In step 410 of the method 400, a set of heartbeat data can be collected. For example, the heartbeat monitor 300′, 310′ can be used to collect a set of heartbeat data for a predetermined amount of time, a predetermined number of samples, an unspecified time or number of samples (e.g., continuous generation of values indicative of heartbeat data), or for a dynamically changing amount of time or number of samples. However, other parameters can also be used to define the set of heartbeat data.

In step 420, a physiological property related to the reliability of a HRV measurement can be collected using the second property detector 320, 320′. In some embodiments, at least a portion of step 410 is substantially concurrent with at least a portion of step 420. In step 430, HRV is calculated from the heartbeat data if the physiological property related to the reliability of a HRV measurement has a predetermined value using the data processor 330, 330′.

As discussed above, in some embodiments, HRV monitor 310, 310′ uses a PPG, or other device, to detect pulsatile volume changes in blood pooling in the capillaries. In certain data collection environments, there is not enough blood pooling to reliably and accurately determine HRV. Specifically, at low skin temperatures, the HRV data is unreliable.

Such low skin temperatures can result from: (1) low ambient temperatures, (2) a physiologic condition, (3) a severe emotional state, or combinations of these effects. In some embodiments, the HRV data is deemed unreliable if the reliability data have a predetermined value, for example, where the skin temperature is less than about 68° F. (20 ° C.).

In other embodiments, additional physiologic signals, for example, respiration rate, can be measured and used as a reliability indicator, either in conjunction with other reliability indicators, or alone.

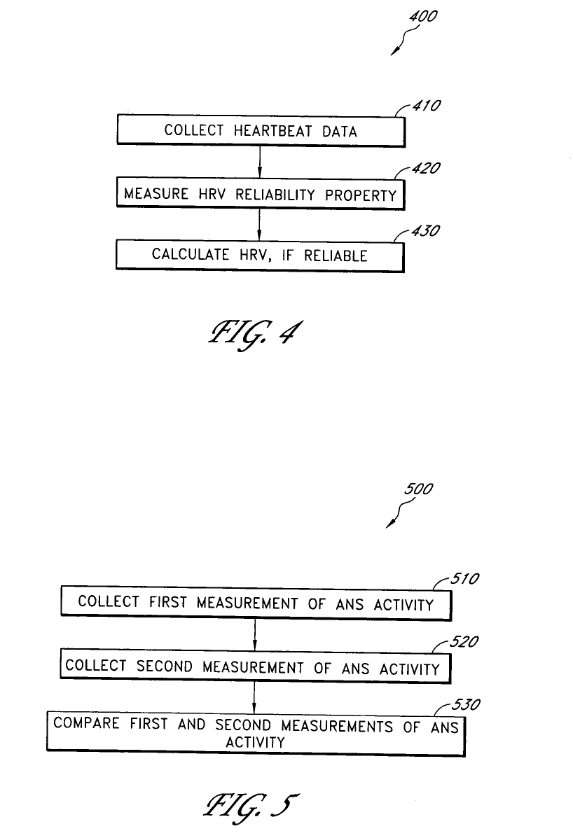

In some embodiments, the monitor 300, 300′ can output a value indicative of the HRV, for example, to a computer (not shown) if the HRV calculation was deemed reliable in the step 420. If the HRV calculation was not deemed reliable in the step 420, then the monitor can simply not output an HRV value, or it can output a message that the HRV cannot be determined, or another message. FIG. 5 is a flowchart illustrating an embodiment of a method 500 for detecting ANS arousal. The following description references the monitors 300, 300′, 300″ described above. Those skilled in the art will understand that the method is applicable to other devices.

In step 510, a first measurement of ANS activity is collected using the second property detector 320, 320′, 320″. In step 520, a second measurement of ANS activity is collected using the second property detector 320, 320′, 320″. In step 530, the first and second measurements of ANS activity are compared using the data processor 330, 330′ to determine whether ANS arousal has occurred.

In this example, ANS arousal is determined by analyzing the SCL and Skin Temperature signals using any of the monitors 300, 300′, 300″. In this case, a baseline activity level for both SCL and Skin Temperature is established. ANS arousal typically results in deviations from the baseline HRV activity. For example, in the case of the SCL, standard signal processing techniques are utilized to isolate the AC component of the SCL, which is the arousal response. This response tends to be a “near instantaneous” response, which is evident within seconds of ANS arousal. Skin temperature changes as a result of ANS arousal tend to be more slow moving and take longer to recover to baseline levels.

After the baseline SCL and skin temperature data are acquired in step 510, a second set of ANS activity data can be collected in step 520.

In step 530, an ANS arousal is determined if either the processed SCL or Skin Temperature changes by a predetermined value. In some embodiments, the first derivative of the SCL conveniently used, and is referred to herein as “skin conductance response” or “SCR.” In some embodiments, a change of 2% in the SCR or a change of 2% in skin temperature indicates ANS arousal. In other embodiments, one or more additional physiologic signals, for example, respiration rate, are measured and used as an ANS arousal indicator.

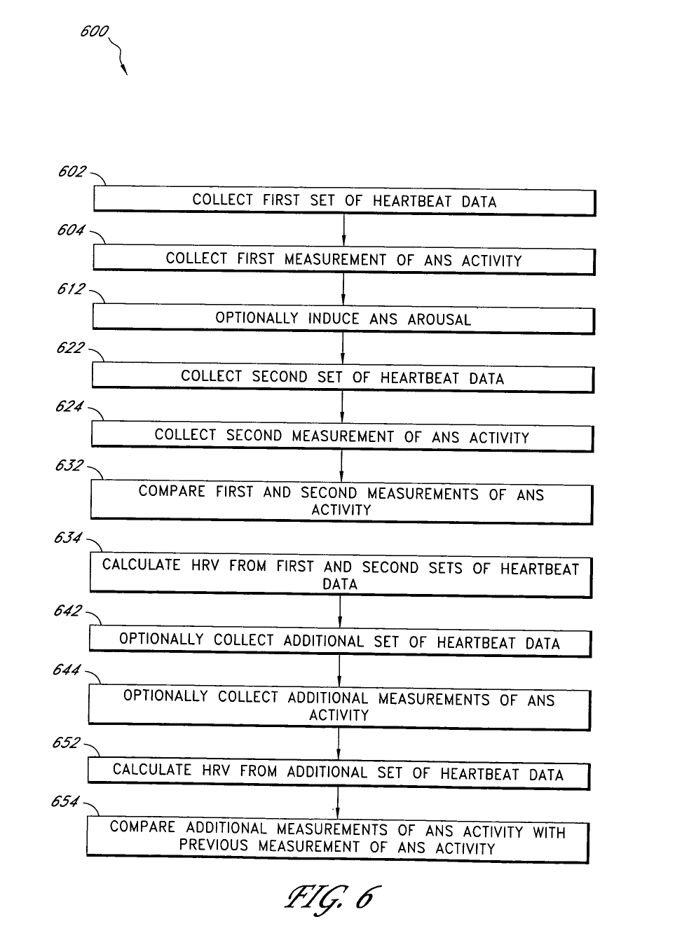

FIG. 6 is a flowchart illustrating an embodiment of a method 600 for determining the effect of ANS arousal on HRV. The following description references the monitor 300 illustrated in FIG. 3A. However, it is to be understood that the other monitors 300′, 300″ can also be used. Those skilled in the art will understand that the method is applicable to other devices as well.

In step 602, a first set of heartbeat data is collected using the heartbeat monitor 310. In step 604, a first measurement of ANS activity is collected using the second property detector 320.

In optional step 612, ANS arousal is induced, as discussed in greater detail below. In step 622, a second set of heartbeat data is collected using the heartbeat monitor 310.

In step 624, a second measurement of ANS activity is collected using the second property detector 320. In step 632, the first and second measurements of ANS activity are compared using the data processor 330. In step 634, HRV are calculated from the first and second sets of heartbeat data. In step 636, the effect of ANS arousal on HRV is determined.

Some embodiments further comprise additional steps. In step 642, at least one additional set of heartbeat data can optionally be collected using the heartbeat monitor 310. In step 544, at least one additional measurement of ANS activity can optionally be collected using the second property detector 320.

In step 552, HRV can be calculated from the at least one additional set of heartbeat data using the data processor 330. In step 654, the at least one additional measurement of autonomic nervous system activity can be compared with a previous measurement of autonomic nervous system activity using the data processor 330.

In some embodiments, at least a portion of step 602 can be performed substantially concurrently with at least a portion of step 604. In other embodiments, steps 602 and 604 are not performed substantially concurrently. Instead, for example, steps 602 and 604 can be performed sequentially. Similarly steps 622 and 624, and 642 and 644, respectively, are independently performed substantially concurrently or sequentially in different embodiments.

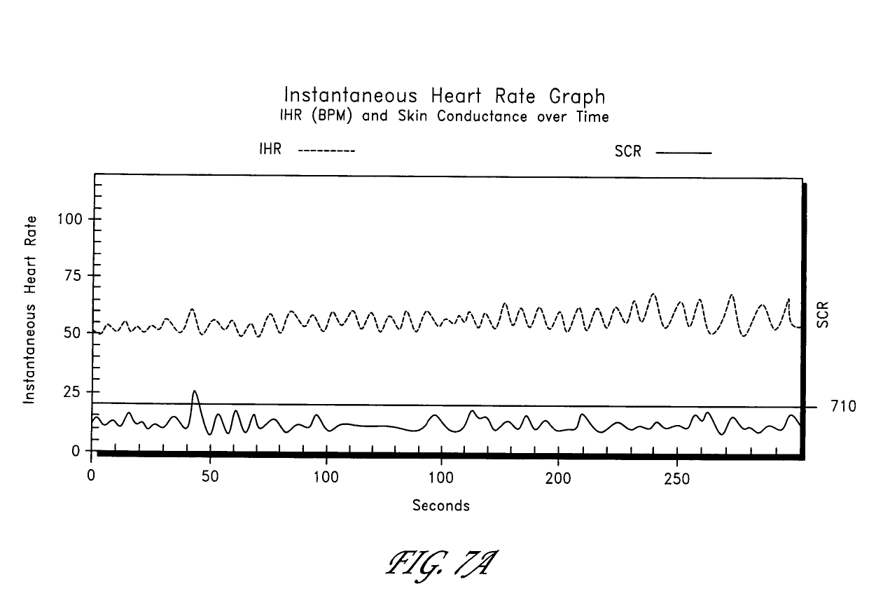

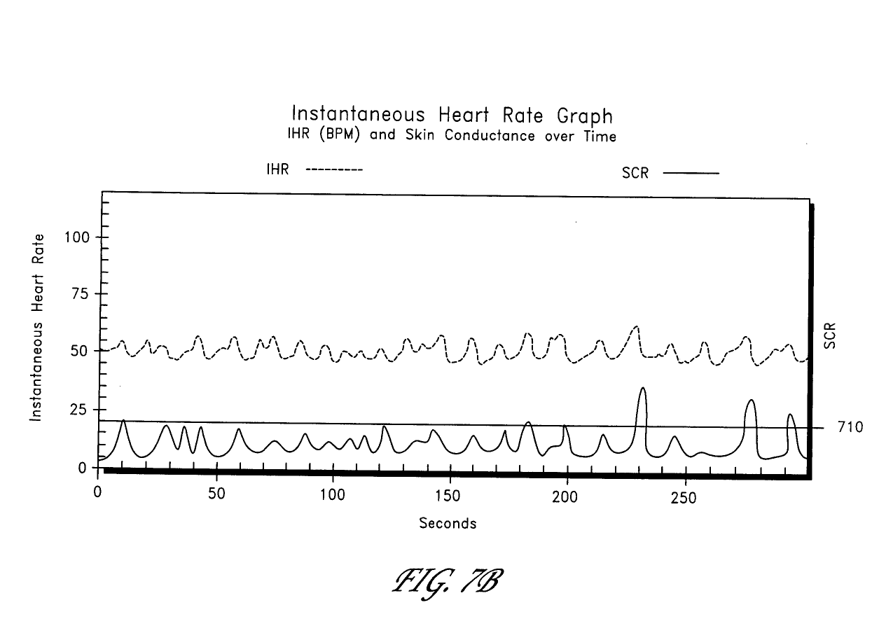

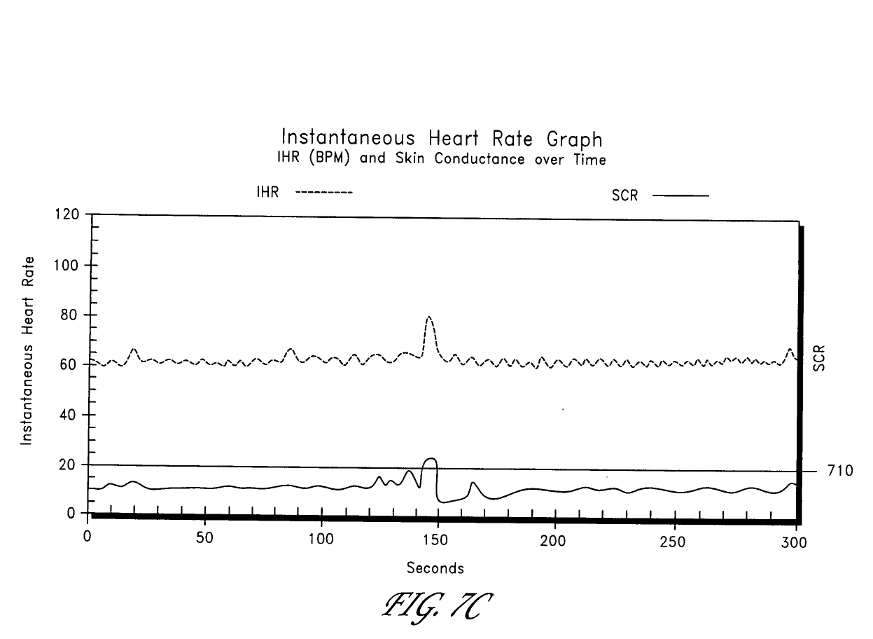

FIGS. 7A-7C illustrate graphs of time series of Instantaneous Heart Rate (IHR) and Skin Conductance Response acquired using any of the monitors 300, 300′, 300″. In the graphs of FIGS. 7A-7C, the IHR is the upper trace and the SCR is the lower trace. Line 710 indicates a level for the SCR indicating ANS arousal. In each graph, where portions of the SCR cross the line 710, the IHR also increases, illustrating the relationship between SCR and heart rate. In the case illustrated in FIG. 7C, the effect is particularly dramatic.

The following Examples illustrate applications of method 500.

### EXAMPLE 1

### Determining the Effects of the ANS Arousal on HRV

In this Example, the precise time window in which an ANS arousal has occurred is determined using method 500 by monitoring SCL and Skin Temperature using any of the monitors 300, 300′, 300″. However, in the description set forth below, it is to be understood that although only the monitor 300 is specifically referred to, the other monitors 300, 300″ can also be used in the same manner described.

First or baseline sets of HRV data and ANS activity data can be collected concurrently in steps 502 and 504, respectively. In steps 522 and 524, second sets of HRV and ANS activity data can be acquired concurrently, and in step 534, the first and second sets of ANS activity data are analyzed to determine if ANS arousal has occurred. If ANS arousal has occurred, in step 542, HRV data for this time window is analyzed and compared to baseline data to determine the effects of ANS arousal on HRV.

Successive time windows can be selected for collection of additional HRV and ANS activity data in steps 542 and 544, respectively, to determine the magnitude and time duration of effect of ANS arousal on HRV in steps 552 and 554. Provided below are exemplary time domain and frequency domain parameters derived from the heartbeat data that are also useful for determining the effect of ANS arousal on HRV. Those skilled in the art will understand that these parameters are useful either alone or in combination.

Heart Rate (HR): The mean heart rate value averaged on entire recording (trial). HR can be measured in beats per minute (BPM).

Mean NN: The mean interbeat interval value averaged on entire recording (trial). Mean NN can be measured in milliseconds.

SDNN: The standard deviation of the NN intervals, which is the square root of their variance. The variance is mathematically equivalent to the total power of spectral analysis, so it reflects all cyclic components of the variability in recorded series of NN intervals. The actual values of SDNN depend on the length of recording; the longer recording is, the higher SDNN values are. Thus, in practice it is preferred to compare SDNN values derived from NN recording of the same length. SDNN can be measured in milliseconds.

RMS-SD: The square root of the mean squared differences of successive NN intervals. This can be indicative of an estimate of high-frequency variations in heart rate in short-term NN recordings that reflects an estimate of parasympathetic regulation of the heart. RMS-SD can be measured in milliseconds.

Total Power: The short-term estimate of the total power of power spectral density in the range of frequencies between 0 and 0.4 Hz. This measure reflects overall autonomic activity where sympathetic activity is a primary contributor. Total Power can be calculated in milliseconds squared (ms²).

Very Low Frequency: The band of power spectrum range between 0.0033 and 0.04 Hz. This measure is not well defined in terms of physiological mechanisms causing VLF component of the power spectrum. Generally it is known this parameter indicates overall activity of various slow mechanisms of sympathetic function. Very Low Frequency band can be calculated in milliseconds squared (ms²).

Low Frequency: The band of power spectrum range between 0.04 and 0.15 Hz. This measure reflects both sympathetic and parasympathetic activity. Generally it can be a strong indicator of sympathetic activity in long-term recordings. Parasympathetic influence can be represented by LF when respiration rate is lower than 7 breaths per minute or during taking a deep breath. Thus, when subject is in the state of relaxation with a slow and even breathing, the LF values can be very high indicating increased parasympathetic activity rather than increase of sympathetic regulation. Low Frequency band can be calculated in milliseconds squared (ms²).

High Frequency: The band of power spectrum range between 0.15 and 0.4 Hz. This measure reflects parasympathetic (vagal) activity. HF is also known as a ‘respiratory’ band because it corresponds to the NN variations caused by respiration (this phenomenon is known as respiratory sinus arrhythmia (RSA)). Heart rate can be increased during inhalation and dropped during exhalation. Slower and even breathing can cause an increase in the amplitude of HF peak on power spectrum. High Frequency as such can be calculated in milliseconds squared (ms²).

LF/HF Ratio: The ratio between the power of Low Frequency and High Frequency bands. This measure can be indicative of overall balance between the sympathetic and parasympathetic systems. Higher values reflect domination of the sympathetic system, while lower ones reflect domination of the parasympathetic system. However, when deep and even breathing occurs, the elevation of this parameter reflects increase of parasympathetic regulation due to effect of RSA. The LF/HF Ratio can be calculated in normalized units.

Normalized Low Frequency: The ratio between absolute value of the Low Frequency and difference between Total Power and Very Low Frequency. This measure minimizes an effect of changes in Very Low Frequency power and emphasizes changes in sympathetic regulation. Normalized LF can be calculated in percentile units.

Normalized High Frequency: The ratio between absolute value of the High Frequency and difference between Total Power and Very Low Frequency. This measure minimizes an effect of changes in Very Low Frequency power and emphasizes changes in parasympathetic regulation. Normalized HF can be calculated in percentile units.

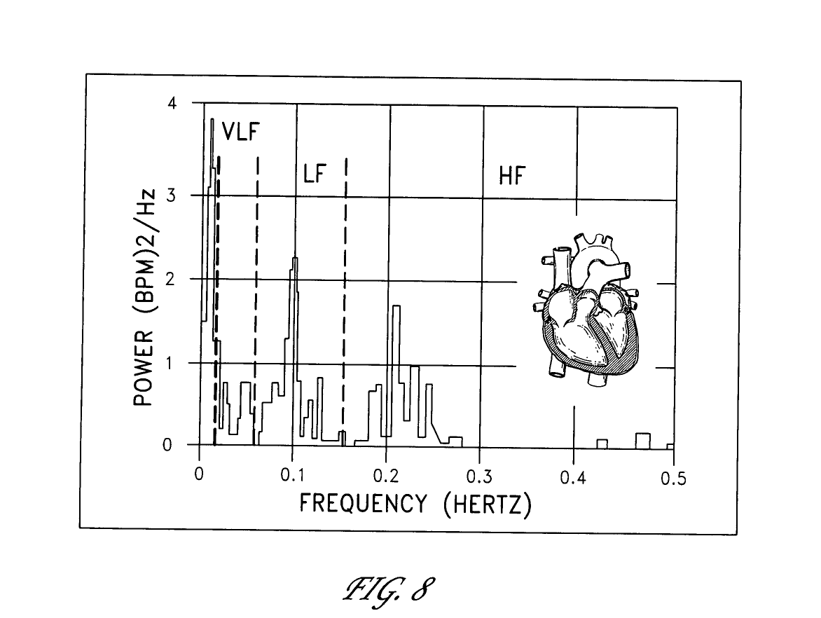

FIG. 8 illustrates the VLF, LF, and HF components of an exemplary set of heartbeat data in (BPM)². In other embodiments, additional time and frequency-based parameters can be monitored, either alone or in combination with at least one of the parameters discussed above. For example, in some embodiments, the heart rate data is transformed to the frequency domain every 32 beats, with a sliding window of 8 beats. A statistical analysis of the VLF, LF, and HF provides a statistical likelihood that these parameters were affected by the arousal. Those skilled in the art will understand that the effect of ANS arousal on other parameters discussed herein are also determinable by statistical analysis.

### EXAMPLE 2

### Determining a Recommended Minimum Extent to Which a Timed HRV Study can be Extended After an ANS Arousal

The time period during which HRV data is affected by ANS arousal can be isolated and determined as discussed above in Example 1. In the present Example, the affected data is not utilized in the HRV data analysis and is eliminated. In order to collect a full data epoch (typically five minutes) in the minimum amount of time, data collected before the ANS arousal and after the end of ANS arousal, for example, as identified in Example 1, are combined and analyzed. In some embodiments, standard signal processing techniques are used to eliminate sections of either or both of the pre-ANS arousal or post-ANS arousal data. The resulting data are aligned such that the frequency content of the combined data is not different from either of the pre- and post-ANS arousal regions. Thus, the recommended extension of time to the standard five minutes can equal the time from the beginning of the ANS arousal until the end of the ANS arousal.

### EXAMPLE 3

### A Generalized Data Analysis Method for Analyzing the Effects of ANS Arousal on HRV

This Example is similar to Example 1, except the subject is subjected to one or more protocols designed to induce one or more ANS arousals. For example, the protocols noted below can be used to cause an ANS arousal for the step 512 of the method 500. Suitable protocols are known in the art, for example, an orthostatic test in which the subject first lies in the horizontal position, and then elevates to the standing vertical position. Another example of such a protocol is one in which the subject is instructed to visualize or describe a traumatic event. By simultaneously monitoring the HRV along with one or more other physiologic parameters, as discussed above, and analyzing the results, the effect of ANS arousal on HRV can be determined. Examples of suitable data analyses include: (1) the post-processing elimination of ANS arousal and the isolation of the effects of basal ANS activity on HRV, (2) the post-processing isolation of the effects on HRV of ANS arousal, and (3) the analysis of recovery of HRV from ANS arousal.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. An apparatus for measuring heart rate variability comprising:
a heartbeat detector; and
a detector configured to detect a physiological property that affects a heart rate variability measurement.

2. The apparatus of claim 1, further comprising a data processor configured to accept data from the heartbeat detector and the detector for a physiological property that affects a heart rate variability measurement, and to calculate a heart rate variability from these data.

3. The apparatus of claim 1, wherein the heartbeat detector comprises a photoelectric plethysmograph.

4. The apparatus of claim 1, wherein the detector configured to detect a physiological property that affects a heart rate variability measurement detects a physiological property that indicate the reliability of an HRV measurement comprising skin temperature, respiration rate, or a combination thereof.

5. The apparatus of claim 1, wherein the detector configured to detect a physiological property that affects a heart rate variability measurement detects a physiological property reflecting ANS activity comprising skin conductance level, skin temperature, or a combination thereof.

6. The apparatus of claim 3, comprising a first and a second detector configured to detect a physiological property that affects a heart rate variability measurement, wherein the first and detectors detect skin conductance level and skin temperature, respectively.

7. An apparatus for determining heart rate variability comprising:
means for measuring heart rate variability;
means for measuring at least one physiological signal, wherein the physiological signal is a reliability indicator for the heart rate variability measurement, an indicator of autonomic nervous system activity, or a combination thereof; and
means for integrating the heart rate variability measurement with the measurement of the at least one physiological signal.

8. A method for determining heart rate variability comprising:
collecting a set of heartbeat data;
detecting a physiological property related to the reliability of a heart rate variability measurement; and
calculating a heart rate variability from the heartbeat data if the physiological property related to the reliability of a heart rate variability measurement indicates that the heart rate variability measurement is reliable.

9. The method of claim 8, wherein the physiological property related to the reliability of a heart rate variability measurement is skin temperature, respiration rate, or a combination thereof.

10. The method of claim 8, wherein at least a portion of the collecting a set of heartbeat data is substantially concurrent with at least a portion of the detecting a physiological property.

11. The method of claim 8, wherein the heart rate variability measurement is deemed reliable when the physiological property related to the reliability of a heart rate variability measurement has a predetermined value.

12. The method of claim 8, wherein the heart rate variability is calculated from at least one of heart rate, mean interbeat interval, standard deviation of the interbeat interval, RMS of the standard deviation of the interbeat interval, total power of the power spectrum, very low frequency component of the power spectrum, low frequency component of the power spectrum, high frequency component of the power spectrum; low frequency to high frequency ratio of the power spectrum, normalized low frequency of the power spectrum, and normalized high frequency of the power spectrum.

13. A method for detecting autonomic nervous system arousal comprising:
acquiring a first measurement of autonomic nervous system activity;
acquiring second measurement of autonomic nervous system activity; and
comparing first and second measurements of autonomic nervous system activity.

14. The method of claim 13, wherein the first and second measurements of autonomic nervous system activity comprise measurements of skin conductance level, skin temperature, or a combination thereof.

15. A method for determining the effect of autonomic nervous system arousal on heart rate variability comprising:
collecting a first set of heartbeat data;
acquiring a first measurement of autonomic nervous system activity;
optionally inducing autonomic nervous system arousal;
collecting a second set of heartbeat data;
acquiring a second measurement of autonomic nervous system activity;
comparing the first and second measurements of autonomic nervous system activity;
calculating a heart rate variabilities from the first and second sets of heartbeat data; and
determining the effect of autonomic nervous system arousal on heart rate variability.

16. The method of claim 15, further comprising:
collecting at least one additional set of heartbeat data;
acquiring at least one additional measurement of autonomic nervous system activity;
calculating a heart rate variability from the at least one additional set of heartbeat data; and
comparing the at least one additional measurement of autonomic nervous system activity with a previous measurement of autonomic nervous system activity.

17. The method of claim 15, comprising inducing autonomic nervous system arousal.

18. The method of claim 15, wherein at least a portion of collecting the first set of heartbeat data is substantially concurrent with at least a portion of acquiring the first measurement of autonomic nervous system activity.

19. The method of claim 15, wherein at least one of the measurements of autonomic nervous system activity is a measurement of skin conductance level, skin temperature, or a combination thereof.

20. The method of claim 15, wherein at least one of the first and second measurements of autonomic nervous system activity indicates a state of autonomic nervous system non-arousal.

21. The method of claim 20, further comprising determining the recovery of heart rate variability from autonomic nervous system arousal.

22. The method of claim 20, further comprising determining a baseline heart rate variability.

23. The method of claim 20, further comprising determining a correction for heart rate variability measurement taken during ANS arousal.

24. The method of claim 16, wherein:
at least two different sets of heartbeat data are aligned and combined, and
a heart rate variability is calculated from the combined sets of heartbeat data.

25. The method of claim 15, wherein the heart rate variability is calculated from at least one of heart rate, mean interbeat interval, standard deviation of the interbeat interval, RMS of the standard deviation of the interbeat interval, total power of the power spectrum, very low frequency component of the power spectrum, low frequency component of the power spectrum, high frequency component of the power spectrum; low frequency to high frequency ratio of the power spectrum, normalized low frequency of the power spectrum, and normalized high frequency of the power spectrum.
